(19) United States
(12) Patent Application Publication
(10) Pub. No.: US 2008/0131332 A1
(43) Pub. Date: Jun. 5, 2008

(54) RFID-based medical equipment sterilization systems and disinfectant dispensers including methods relating thereto

(76) Inventors: Hap Nguyen, Westminster, CA (US); Bich-Dao Thi Nguyen, Irvine, CA (US)

Correspondence Address: Donald Bollella;DB Technical Consulting, 21 Vetrina, Irvine, CA, 92606, US

(21) Appl. No.: 11/983,053

(22) Filed: Nov. 6, 2007

Related U.S. Application Data
(60) Provisional application No. 60/857,076, filed on Nov. 6, 2006.

(51) Int. Cl. A61L 2/24

(52) U.S. Cl. 422/119

(57) ABSTRACT

A disinfectant dispensing system includes a dispensing device implemented to dispense a predetermined amount of disinfectant. The system further includes means for detecting the presence of a user within the vicinity of the system, means for detecting use of the dispensing device, and means for recording the presence of a particular user within the vicinity of the system and use of the dispensing device by said user. A related system for equipment cleaning includes equipment having embedded RFID tags associated with cleaning devices with corresponding RFID readers. Various methods of using the systems are also included.

### CROSS-REFERENCE TO RELATED APPLICATIONS

The present application claims the benefit of priority from U.S. Provisional Application Ser. No. 60/857,076 filed Nov. 6, 2006 which is herein incorporated by reference in its entirety.

### BACKGROUND OF THE INVENTION

1. Field of the Invention

This invention relates in general to medical equipment sterilization and disinfectant dispensing systems. In particular, the invention is directed to sterilization systems including equipment having embedded RFID tags and to disinfectant dispensing systems including RFID tags, bar code readers, magnetic readers, or bio-metric input devices to identify users of the system.

More specifically, but without restriction to the particular embodiments hereinafter described in accordance with the best mode of practice, this invention relates to medical equipment sterilization systems including database monitoring and control of equipment having embedded RFID tags and to disinfectant dispensing systems and methods that employ identification of users of the system to reduce propagation of germs that cause illnesses.

2. Background Discussion and Related Art

It is now well established that germs cause illness. This, however, wasn't always known to be true. In 1847, the Hungarian physician Ignaz Semmelweis was working in a Viennese, maternity hospital with two separate clinics. In one clinic babies were delivered by physicians, and in the other deliveries were handled by midwives. The mortality rate in the doctors' clinic was nearly triple the rate in the midwives' clinic. Semmelweis wondered what caused this huge discrepancy. After investigating, it turned out that the doctors often came to deliveries straight from the autopsy ward, promptly infecting mother and child with whatever germs their most recent cadaver happened to carry. Once Semmelweis had these doctors wash their hands with an antiseptic solution, the mortality rate in the physicians' clinic decreased significantly.

In current times, the Semmelweis discovery of simply having medical personnel wash their hands frequently to reduce the spread of illness-causing germs, as crucial and obvious as it seems, has proven difficult to enforce. A multitude of recent medical studies have shown that hospital personnel including doctors, nurses, and aides; wash or disinfect their hands in fewer than half the instances they should. Post surgical infections caused by medical personnel in hospitals due to non-compliance with professional hygiene standards is still today one of the sore spots of the medical profession.

In another area where hand washing is critical to public health and safety, the Centers for Disease Control and Prevention (CDC) has recently estimated that 76 million cases of food-borne illnesses, or food poisoning, occur every year in the United States. Food-borne illnesses are defined as toxic or infectious diseases caused by agents that enter the body through the ingestion of food. Every person is at risk of food-borne illness, regardless of their health, age, or social stature.

Bacterial pathogens are the leading cause of food poisoning in humans. Food may be contaminated with microorganisms such as E. coli, Salmonella, Listeria, Shigella, or Campylobacter. Other causes of food poisoning may include natural poisons, parasites, harmful chemical substances, or viruses such as Hepatitis A. In the United States, illnesses caused by food-borne contaminants are estimated to cost up to $35 billion annually in medical costs and lost productivity.

The safety of the food we eat has recently drawn attention as a national health dilemma. Despite notable advances in food processing and manufacturing, food-borne illnesses continue to be a significant, and growing, public health problem in the United States and other countries around the world.

In response to increasing incidents of food-borne illness nationwide, organizations such as the CDC are taking action to educate the public about the threat of food-borne illness, and to ensure that the food industry takes steps to protect consumers from food contamination.

Salmonella Enteritidis Infection: Egg-associated salmonellosis is an important public health problem in the United States and several European countries. A bacterium, Salmonella enteritidis, can reside inside perfectly normal-appearing eggs, and if the eggs are eaten raw or undercooked, the bacterium can cause illness. During the 1980s, illness related to contaminated eggs occurred most frequently in the northeastern United States, but now illness caused by S. enteritidis is increasing in other parts of the country as well. Consumers should be aware of the disease and learn how to minimize the chances of becoming ill. To reduce the risk of Salmonella enteritidis infection, the CDC recommends, inter alia, keeping eggs refrigerated, discarding cracked or dirty eggs, and washing hands and cooking utensils with soap and water after contact with raw eggs.

E. Coli Infection: E. coli, the common abbreviation for Escherichia coli, is a bacterial pathogen which exists in cattle and other animals. Humans are usually infected with this bacterium after consuming food or water that has been contaminated with traces of cow feces. Most E. coli infections originate from eating undercooked or raw ground beef, but the bacterium may be present in other foods as well. In 2005, a prominent food company recalled 250,000 bags of pre-cut salads after shoppers in Minnesota became ill with E. coli infection. E. coli is estimated to affect 73,000 people per year with 2,000 being hospitalized and 60 people dying from the infection. The symptoms of E. coli infection are more severe in adults, the elderly, or those with pre-existing disease or illness. E. coli can be easily spread from one person to another, especially when proper hand washing is not practiced. Facilities where this germ is likely to spread include, for example, daycare facilities, schools, and nursing homes. Those infected with E. coli O157:H7 are extremely contagious.

Hepatitis A: Hepatitis A is caused by the hepatitis A virus (HAV). Hepatitis A is a food-borne illness that is spread from person to person, often due to lack of proper hygiene habits such as hand washing. Hepatitis A is typically spread through the consumption of food contaminated with the virus. An average of 80,000 people contract Hepatitis A each year, with about 100 people dying from acute liver failure. In November 2003, green onions originating from Mexico were said to cause an outbreak of Hepatitis A which affected 575 people in Pennsylvania.

One of the simplest and most effective ways to reduce the risk of spreading any of these illnesses, whether transferred by food handling or medical personal, is washing. Thus in the food handling, packaging, and preparation industries having employees washing hands thoroughly and frequently during operations is highly recommended. In the restaurant business in particular, washing hands, counters, and utensils thoroughly after they contact raw beef can have a significant impact on reducing the risk of spreading food-borne illnesses. In the medical and related caretaking professions, having doctors, nurses, aids, and other caregivers wash their hands frequently has proven to have a significant effect on reducing the spread of germs that cause illness. Thus in facilities such as hospitals, daycare centers, schools, and nursing homes; implementing and maintaining an employee hygiene policy that includes frequent hand washing is desired to reduce the risk of spreading germs that cause disease.

Virtually any facility, institution, or profession where employed personnel have frequent contact with the public or food that the public consumes, may benefit from instituting and maintaining an employee hygiene policy that includes frequent hand washing to reduce the risk of spreading germs that cause illness. Such facilities and institutions include, for example, blood handling facilities; public healthcare facilities; pathology labs; doctors' and dentists' offices; hospitals and clinics; schools and daycare centers; nursing homes; restaurants and food preparation areas on ships, airplanes, and trains; university food services, medical facilities, and research labs; all types of food processing plants including meat packing plants, poultry farms and related processing areas, and vegetable or fruit processing plants; farming areas including portable restrooms stationed at field-side packing stations; and all kitchens or restrooms associated with any such facilities.

Prior hereto, there has not been proposed a comprehensive, closed-loop technology-based system or method for effectively implementing an employee hygiene policy focused on hand washing to thereby easily and cost-effectively substantially reduce the risk of spreading germs that cause illness. Thus the inventors hereof disclose below the details of various preferred embodiments of such a system and related methods with the view that health and wellness world-wide may be significantly improved.

### SUMMARY AND OBJECTS OF THE INVENTION

The present invention is directed to ID based disinfectant dispenser systems and methods relating thereto. The present systems and methods are readily adapted to accommodate any type of particular disinfectant including soap-based, alcohol-based, or other types of disinfectants and may be readily implemented in any physical infrastructure or building facilities to accommodate the particular needs of any type of work place environment including, for example, food services, health care, food processing, medical research, farming, educational, and any such environment where employed personnel have frequent contact with the public or food products that the public ultimately consumes.

As the world's population increases and as local or regional population densities increase even further, the practice of proper hygiene becomes more important for maintaining the health and wellness of the general public. In just one industry sector such as tourism, for example, improving and maintaining the health and wellness of tourists traveling through national restaurants, hotels, train stations, cruise ships, and airports is in the economic interests of international business and national governments. Food industry, healthcare, daycare, school, and nursing home workers are supposed to practice proper hygiene habits. There are, however, those who do not comply with the practice, and there are regions of the world where such standards are minimal or completely absent. Such lack of compliance or standards does, at times, put the general public at grave risk of serious infection that could, otherwise, be easily prevented. The present invention is thus directed to and includes a computerized ID based disinfectant dispenser that networks with a server to track who uses the devices, and in certain preferred embodiments when and where (what dispenser number) the devices are used, to thereby ensure that relevant health code requirements are complied with.

The employee data base in the server captures the data on the workers' RFID tags or other ID input device and their activities at these networked dispensers. With this collection of information, the employers can then take appropriate actions to remedy any compliance problems. This aspect of the present invention is also readily applied to hand equipment in any industry and particularly in the medical profession as applied to medical equipment and the need for sterilization.

In one preferred embodiment relating to hand washing, each employee is issued a passive RFID tag to wear on his or her wrist. When the so-outfitted employee finishes a visit to the bathroom or brings his or her hand underneath the dispenser (where the RFID reader is embedded), the dispenser will automatically dispense the disinfectant. In this manner, a record is made in the server data base that the employee has complied with a hand washing policy. In further embodiments of the system aspects of the present system, the entry door of the wash area is equipped with a sensor that detects the entry of an employee into the washroom area. In this manner, in the event that an employee enters a wash room area and does not use the disinfectant dispenser according to the present invention, a record will be made of such non-use. In still further embodiments of system aspects of the present invention, a foot-activated door knob is utilized to eliminate any contact with the doorknob after the employee's hands have been sanitized.

While there may be those in the commentary who would criticize the present invention on the basis of certain privacy issues, the inventors hereof believe that any perceived or argued curtailment of personal freedoms attributed to implementation or practice of the present systems or methods is far outweighed by the increase in health and wellness that the present inventions will bring to the general public.

It is, therefore, an object of the present invention to improve the health and wellness of the general public.

Another object of this invention is to utilize a closed-loop system to ensure that employers are in compliance with health code requirements relating to employee hygiene.

Still yet a further object of the present invention is to employ a closed-loop system to ensure that employers are in compliance with health code requirements relating to equipment cleanliness whether used in the food industry, medical profession, or any other area where unwashed or cleaned hand-held tools or equipment may contribute to the spread of illness.

These and other objects are attained in accordance with the present invention wherein there is provided, inter alia, a disinfectant dispensing system that includes a dispensing device implemented to dispense a predetermined amount of disinfectant. The system includes means for detecting the presence of a user within the vicinity of the system, means for detecting use of the dispensing device, and means for recording the presence of a particular user within the vicinity of the system and use of the dispensing device by said user. Various methods of using the system are also included.

The present invention is further directed to an equipment disinfecting and tracking system. This system includes an equipment cleaning apparatus, means for detecting the presence of a particular piece of equipment within the vicinity of said equipment cleaning apparatus, means for detecting application of equipment cleaning apparatus to said particular piece of equipment, and means for recording the presence of said particular piece of equipment within the vicinity of said equipment cleaning apparatus and use thereof by a user. Various methods of using this system are also included.

### BRIEF DESCRIPTION OF THE DRAWING FIGURES

Further objects of the present invention together with additional features contributing thereto and advantages accruing therefrom will be apparent from the following description of certain preferred embodiments of the invention which are shown in the accompanying drawing with like reference numerals indicating like components throughout, wherein:

FIG. 1 is a partially broken-way perspective view of a washroom equipped with various aspects of the present invention showing for purposes of illustration a chef using a disinfectant dispensing device for hand washing according to the teachings hereof;

FIG. 2A is a pictorial cut-away view of the disinfectant dispenser of the present invention shown in conjunction with a block diagram representing various aspects, characteristics, and functionalities of the present system as integrated with the illustrated dispenser;

FIG. 2B is a schematic diagram of an equipment cleaning device such as an autoclave in conjunction with a block diagram representing various aspects, characteristics, and functionalities of the system thereof and methods relating thereto;

FIG. 3 is an enlarged perspective pictorial view of the disinfectant dispenser according to the present invention including alternate ID input devices such as a thumb print scanner, barcode scanner, magnetic card reader, or iris scanner as illustrated;

FIG. 4 is a detailed block diagram of alternate preferred embodiments of the system of the present invention as may be implemented in any desired work environment to achieve the intended purposes thereof;

FIG. 5 is a logic flow chart representing the principal steps of one preferred embodiment of a method of operating a dispensing system according to the present invention;

FIG. 6 is an enlarged perspective pictorial view of the present disinfectant dispenser with the alternate ID input devices of FIG. 3 further including an additional controller input for implementing a semi-automatic mode according an alternate embodiment of the system aspects of this invention; and

FIG. 7 is a logic flow chart representing the principal steps of another preferred embodiment of a method of operating a dispensing system according to the semi-automatic aspects of present invention illustrated in FIG. 6.

### DETAILED DESCRIPTION OF THE PREFERRED EMBODIMENTS

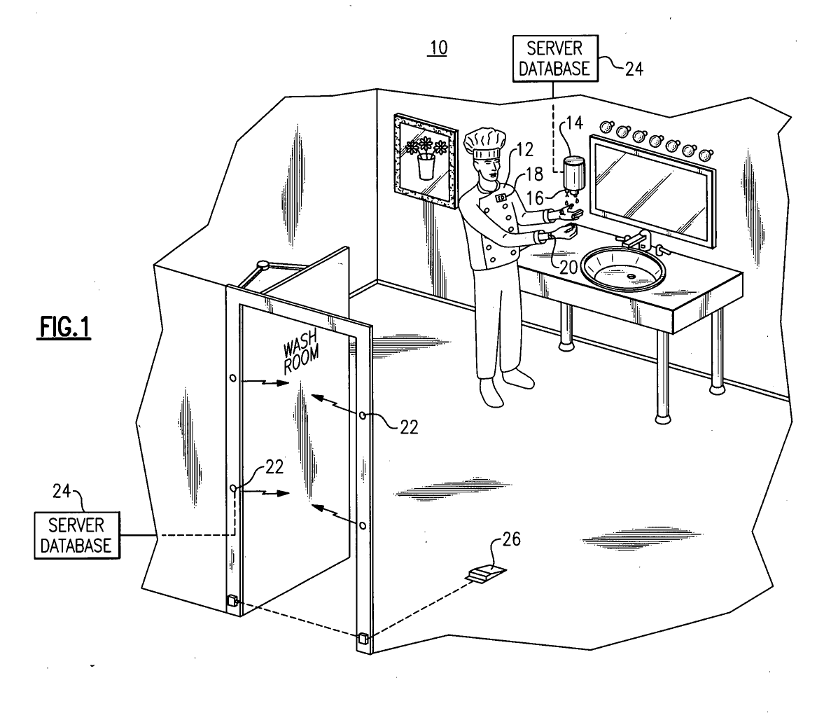

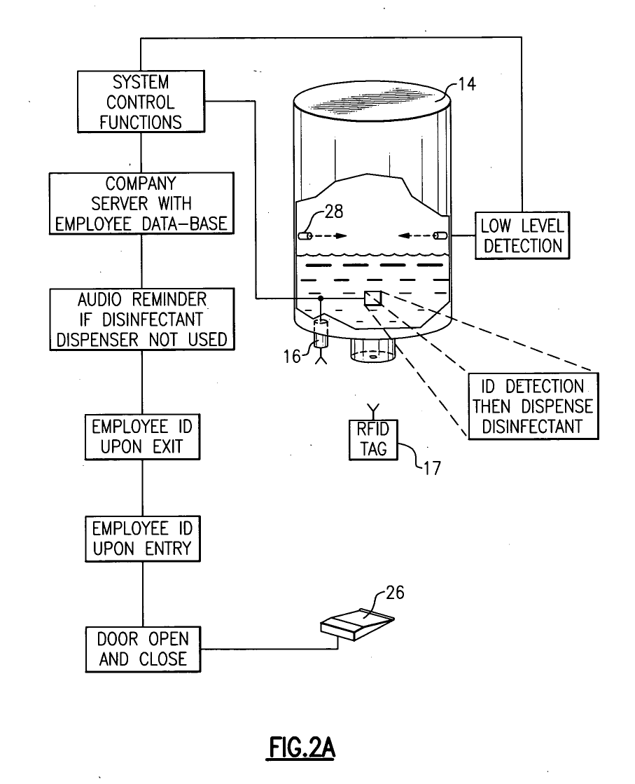

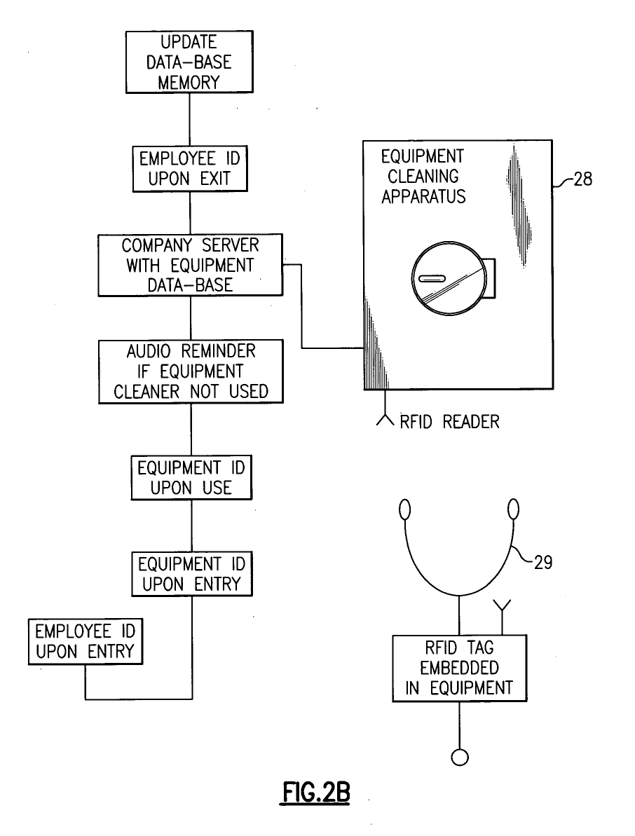

Referring now to FIG. 1 there is shown a preferred embodiment of a disinfectant dispensing system in accordance with the present invention. More particularly, FIG. 1 is a partially broken-way perspective view of a washroom 10 equipped with various aspects of the present invention showing for purposes of illustration an employee 12, in this case a chef, using a disinfectant dispensing device 14 for hand washing according to the teachings hereof. In this specific embodiment, the disinfectant dispensing device 14 includes an RFID transponder 16 that receives an RFID signal from an RFID tag 17 (FIG. 2A) embedded in either an employee ID badge 18 or an employee issued ID bracelet 20.

The washroom 10 illustrated in FIG. 1 is further provided with RFID readers 22 positioned, for example, in the entrance door as shown. The RFID readers 22 in the entrance door and the RFID transponder 16 in the disinfectant dispensing device 14 are both connected to a server and database 24 according to a principal aspect of the present invention. In this manner, when the employee enters the washroom 10, the readers 22 record his entry. This information is saved in the server database 24. As the employee 12 proceeds with use of the washroom, he approaches the disinfectant dispensing device 14 to obtain a shot or discharge of an amount of disinfectant for washing his hands. As he approaches the disinfectant dispensing device 14, the RFID transponder 16 reads his personal employee ID as transmitted by the RFID tag embedded in either the employee ID badge 18 or an employee issued ID bracelet 20. This use of the disinfectant dispensing device 14 is further recorded in the server database 24 as represent in FIG. 1. The washroom 10 illustrated in FIG. 1 is further equipped with and automatic door opener 26, here shown as a foot peddle switch as being illustrative of one of several possible implementations. The automatic door opener allow the employee to exit the wash room Thus in this manner, proper use of the disinfectant dispensing device 14 by the employee is recorded in the company server and database 24.

With reference now to FIG. 2A, there is shown a pictorial cut-away view of the disinfectant dispenser 14 of the present invention shown in conjunction with a block diagram representing various aspects, characteristics, and functionalities of the present system as integrated with the illustrated dispenser. In this embodiment, in the event the employee exits the washroom 10 without having used the dispenser 16, the RFID readers 22 in the entrance/exit door will detect the exit without having recent operation of the dispenser associated that that particular employee ID. In this case, the system is equipped with an audio output device such as sound speakers that then give an audible sound or voice reminder that the employee is exiting the washroom without having used the dispenser 14. In some critical situations such as hospital operating rooms, the washroom doors may be automatically disabled or locked by the system controller until the employee has properly used the dispenser 14 and that use is recorded by the system controller. FIG. 2A further illustrates that the dispenser 14 may be equipped with a low level detection device 28 to detect when the level of disinfectant in the dispenser has reached a low point where re-filling is required. The low level detection device 28 is connected to the system controller as illustrated so that a signal may be sent to janitorial services indicating the dispenser requires re-filling. In a system where several dispensers 14 are employed, each is given a serial number and location so that when the low level signal is received at the system controller, the serial number and location of the dispenser can be made known to the janitorial services personnel.

With reference now to FIG. 2B, there is shown a schematic diagram of an equipment cleaning device 28 such as an autoclave and a block diagram representing various aspects, characteristics, and functionalities of the system implementation thereof and methods relating thereto. There is also shown a piece of equipment, here by way of example a doctor's stethoscope 29, which includes an embedded RFID tag. As in the case of the hand-washing system above, the company server including an indexed equipment database may be updated when a piece of equipment such as the stethoscope 29 is properly disinfected by use of the autoclave. As in the case above, the room containing the equipment cleaning device 28 may be outfitted with RFID readers 22 to determine when equipment needing cleaning has entered the room containing the cleaning device 28. And similarly, an employee charged with the duty of cleaning the equipment may be identified by his RFID employee badge upon entry and exit of the room containing the cleaning device 28.

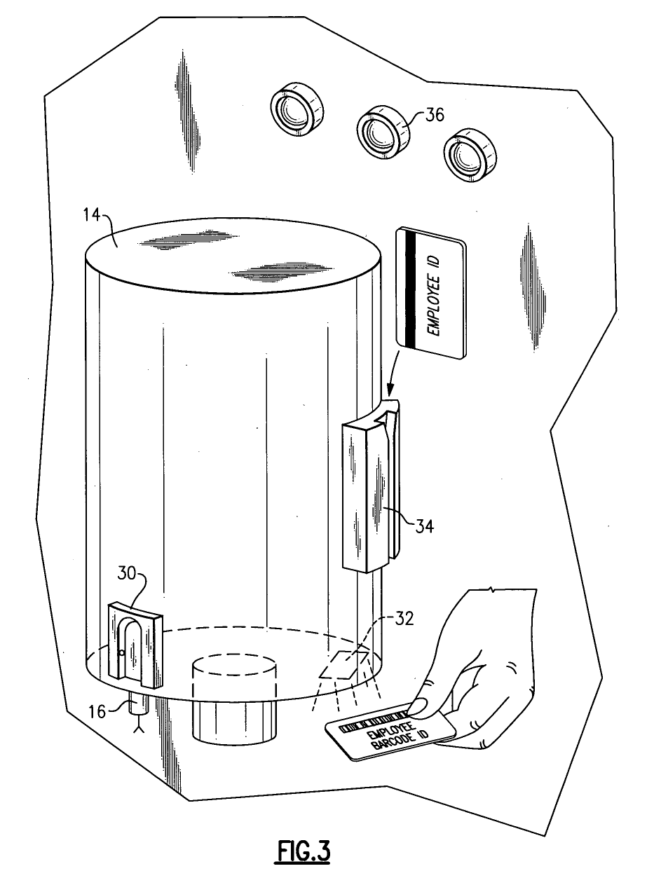

FIG. 3 is an enlarged perspective pictorial view of the disinfectant dispenser according to the present invention including alternate ID input devices such as a thumb print scanner 30, barcode scanner 32, magnetic card reader 34, or iris scanner 36 as illustrated. These alternate input devices may be employed alone in individual cases or in combination with the RFID employee tags and reader discussed above.

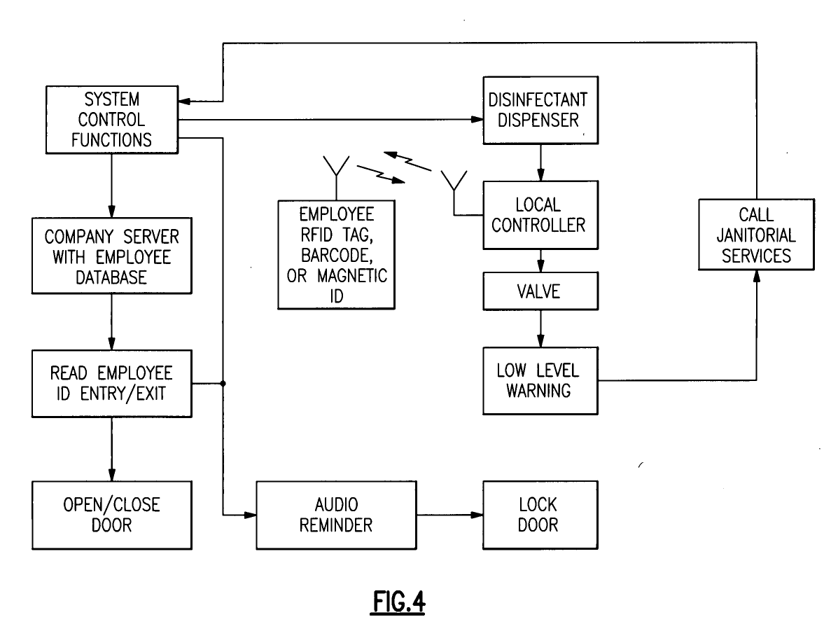

FIG. 4 is a detailed block diagram of alternate preferred embodiments of the system of the present invention as may be implemented in any desired work environment to achieve the intended purposes thereof. Here there is shown an employee RFID tag, barcode, or magnetic ID in communication with a local controller. This system is not limited to implementation in a washroom but may be readily adapted to any hand washing environment including, for example, blood handling facilities; public healthcare facilities; pathology labs; doctors' and dentists' offices; hospitals and clinics; schools and daycare centers; nursing homes; restaurants and food preparation areas on ships, airplanes, and trains; university food services, medical facilities, and research labs; all types of food processing plants including meat packing plants, poultry farms and related processing areas, and vegetable or fruit processing plants; farming areas including portable restrooms stationed at field-side packing stations; and all kitchens or restrooms associated with any such facilities. The inventors hereof believe that the system and methods illustrated in FIG. 4 may be readily adapted to the equipment cleaning system shown in FIG. 2B by one of skill in the art without undue experimentation.

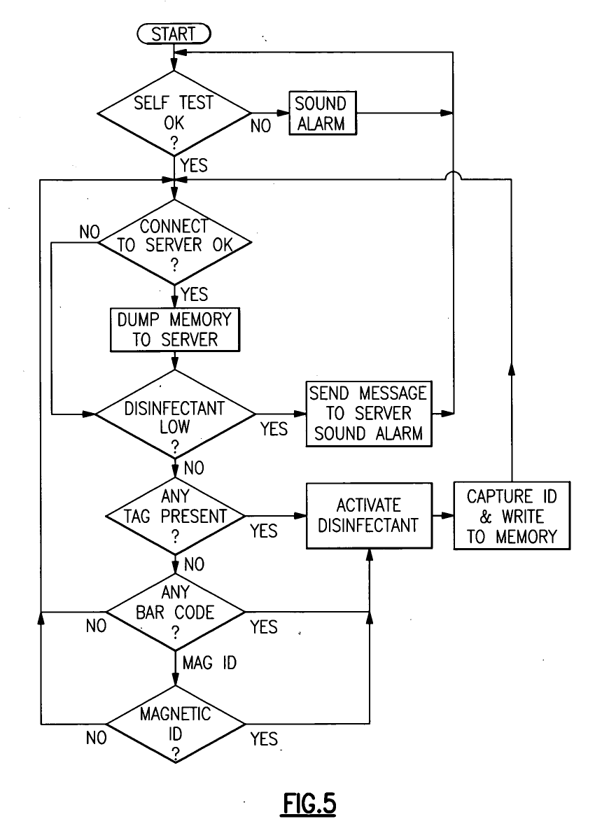

FIG. 5 is a logic flow chart representing the principal steps of one preferred embodiment of a method of operating a dispensing system according to the present invention. This method includes the steps of conducting a self test, connecting to the company server, dumping memory to the server, checking if the disinfectant level is low, and checking for the presence of an RFID tag, bar code, or magnetic ID. In the case where the self test is negative, the method proceeds to the step of sounding an alarm. And similarly, if the disinfectant is low a message is sent to the server to sound the alarm. In this manner, a quick disinfectant fluid refill may be performed. In the case where the method proceeds normally, after the detection of an RFID tag, bar code, or magnetic ID, the method proceeds to activating or dispensing the disinfectant, capturing or logging the ID, and writing or updating the system memory accordingly.

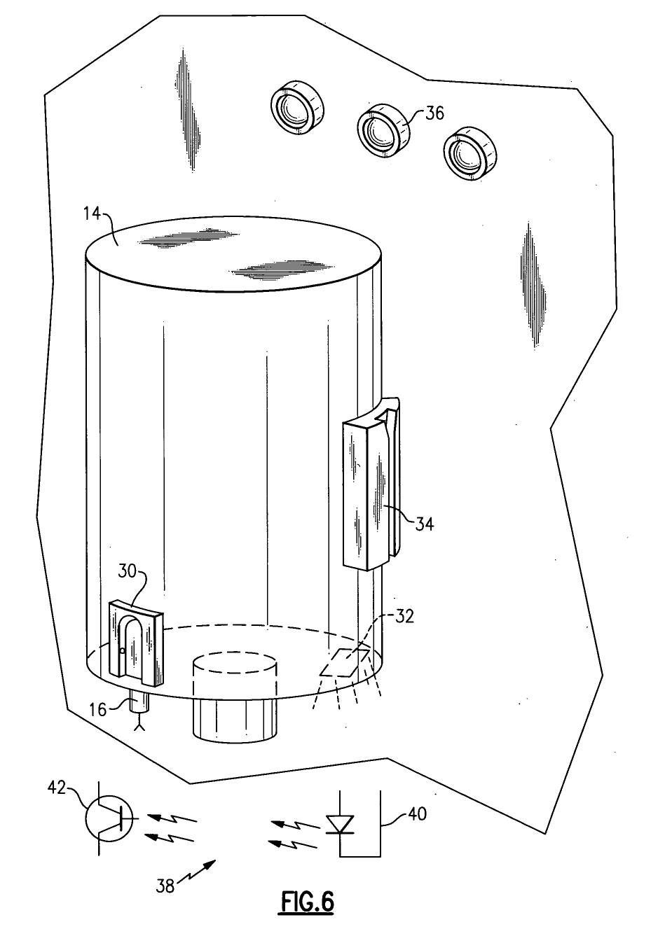

FIG. 6 is an enlarged perspective pictorial view of the present disinfectant dispenser 14 with the alternate ID input devices of FIG. 3 further including an additional controller input here shown in the form of a photo interrupter 38 for implementing a semi-automatic mode according an alternate embodiment of the system aspects of this invention. The photo interrupter 38 as here illustrated by way of example includes a photo diode 40 and a photo transistor 42. The photo diode 40 and the photo transistor 42 are in optical communication with each other such that a beam of light is maintained therebetween. The wavelength of the light may be of any desired length and is not limited to wavelengths in the visible range. Use of the photo diode 40 and the photo transistor 42 in this particular embodiment are by way of example since there would be many alternates of implementing this technical aspect of the present invention as would be apparent to one of skill in the art given the present disclosure. Alternates would include any type of proximity detectors or motion sensors as are well know and currently available as discrete or individual components. The circuit containing the photo diode 40 and the photo transistor 42 is connected in association with the local controller illustrated in FIG. 4. Thus in this manner, liquid disinfectant is only dispensed when the light beam is broken and a proper ID is received by any one of the ID inputs of the disinfectant dispensing device 14. A limited time delay between the receipt of the ID and beam interruption may be advantageously programmed into the controller.

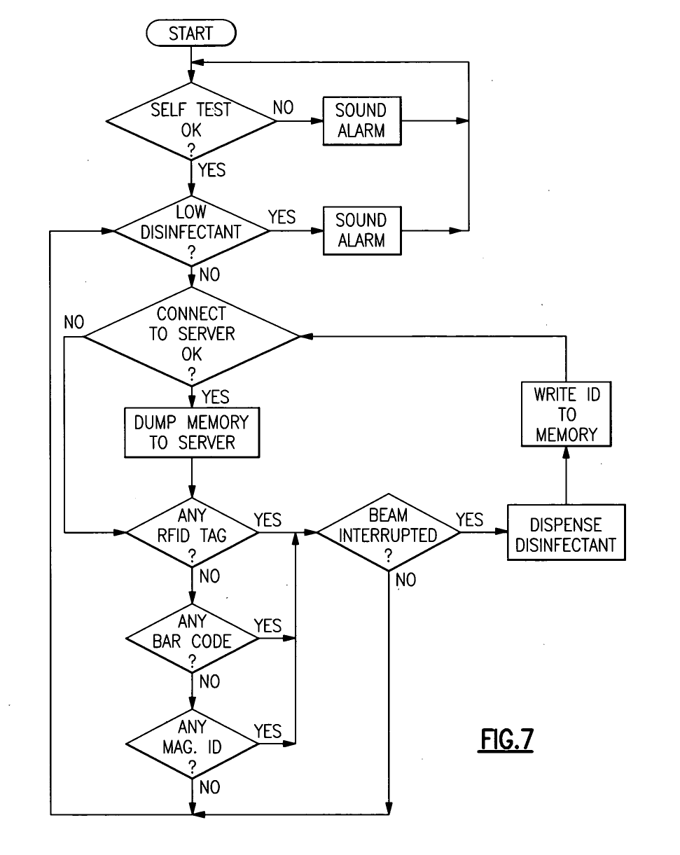

FIG. 7 is a logic flow chart representing the principal steps of another preferred embodiment of a method of operating a dispensing system according to the semi-automatic aspects of present invention illustrated in FIG. 6. This method proceeds in a manner similar to the method described in connection with FIG. 5, and as illustrated includes implementation of the photo interrupted 38. Thus the method here includes the further steps of checking for any authorized ID input from an RFID tag, bar code, or magnetic tape, and then the further step of inquiring whether the beam between the photo diode 40 and the photo transistor 42 has been interrupted. If yes, and any time limitation is met, then a predetermined shot of liquid disinfectant is automatically dispensed. Thereafter, the method proceeds to record the ID and use in the system memory. As with the above systems, the use record may include date, time, and location of the device as well as the employee ID.

While this invention has been described in detail with reference to certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

What is claimed is:

1. A disinfectant dispensing system, comprising:
a dispensing device implemented to dispense a predetermined amount of disinfectant;
means for detecting the presence of a user within the vicinity of the system;
means for detecting use of the dispensing device by a particular user; and
means for recording the presence of said user within the vicinity of the system and use of the dispensing device by said user.

2. An equipment disinfecting and tracking system, comprising:
an equipment cleaning apparatus;
means for detecting the presence of a particular piece of equipment within the vicinity of said equipment cleaning apparatus;
means for detecting application of said equipment cleaning apparatus to said particular piece of equipment; and
means for recording the presence of said particular piece of equipment within the vicinity of said equipment cleaning apparatus and use thereof by a user.
